(19) United States
(12) Patent Application Publication
(10) Pub. No.: US 2019/0008672 A1
(43) Pub. Date: Jan. 10, 2019

(54) JOINT FOR AN ORTHOPAEDIC DEVICE

(71) Applicant: OTTO BOCK HEALTHCARE GMBH, Duderstadt (DE)

(72) Inventors: David HOCHMANN, Steinfurt (DE); Marcus LURSSEN, Gottingen (DE); Matthias SCHILLING, Weissenborn-Luderode (DE)

(73) Assignee: OTTO BOCK HEALTHCARE GMBH, Duderstadt (DE)

(21) Appl. No.: 15/747,041

(22) PCT Filed: Jul. 27, 2016
(86) PCT No.: PCT/EP2016/067937

§ 371 Date: Jan. 23, 2018

(30) Foreign Application Priority Data
Jul. 28, 2015 (DE) 102015112283.9

(51) Int. Cl. A61F 5/01; A61F 2/64; A61F 2/66; F16F 3/06

(52) U.S. Cl. CPC A61F 5/0123; A61F 2/64; A61F 2/6607; A61F 2005/0165; A61F 5/0127; A61F 2005/0179; F16F 3/06

(57) ABSTRACT

A joint for an orthopaedic device, in particular an orthosis or prosthesis, wherein the joint has a first element, at least one spring element and a second element, which is mounted so as to pivot on the first element against a force applied by the at least one spring element in at least one direction. The at least one spring element has at least two helical springs which in each case are wound from a spring strip having a longer cross-sectional side upright with respect to the spring axis and are screwed into one another in such a way that the longer cross-sectional side of at least one of the helical springs has an angle different from 90° relative to the spring and that the spring strips butt against one another.

The invention relates to a joint for an orthopedic device, in particular an orthosis or a prosthesis, wherein the joint has a first element, at least one spring element, and a second element which is mounted pivotably on the first element counter to a force applied by the at least one spring element in at least a first direction. A joint of this kind is known, for example in the form of an ankle joint for a leg orthosis, from DE 10 2010 014 334 A1. Ankle joints of this kind can be used in leg orthoses or below-knee orthoses. For therapeutic reasons, it may be expedient to limit the length of the pivoting movement, i.e. the maximum permissible pivoting angle, of the second element relative to the first element and, for example, to provide a stop in one or both directions of pivoting. To avoid too hard an impact on these stops, the latter are generally spring-loaded and therefore damped. This spring-damping additionally ensures that a pivoting of the joint for the orthopedic device is possible only when the force applied by the spring is overcome. This may also be expedient for rehabilitation and training purposes.

Particularly when the joint is used as an ankle joint, but also in other fields of use, the spring element must have a sufficiently high spring force and spring constant while at the same time requiring the smallest possible installation space. In the embodiment known from the prior art, this is achieved by means of a disk spring arrangement, which is designed in particular as a stack of disk spring elements arranged one above another. They have a high spring force and, compared to conventional leaf springs or helical springs of the same spring strength, take up a relatively small installation space. However, a disadvantage is that disk spring arrangements are cost-intensive and, moreover, are complicated to produce and assemble. There is also the danger of one or more of the disk springs breaking, for example under too high a load or on account of fatigue. This would lead to an abrupt reduction of the spring force and therefore of the damping of the joint, as a result of which the person wearing the orthosis in which the joint is fitted could get a fright and, in the worst case, could stumble.

The object of the invention is therefore to further develop a joint of the type in question such that the described disadvantages are reduced or completely eliminated.

The invention achieves the stated object by making available a joint of the type in question for an orthopedic device, in particular an orthosis or a prosthesis, wherein the joint has a first element, at least one spring element, and a second element which is mounted pivotably on the first element counter to a force applied by the at least one spring element in at least a first direction, wherein the joint is characterized in that the at least one spring element has at least two helical springs which are each wound from a spring strip having a longer cross-sectional side edgeways with respect to the spring axis and which are screwed into each other in such a way that the longer cross-sectional sides have an angle deviating from 90° relative to the spring axis in different directions, and the spring strips bear on each other.

Spring elements of this kind are sold, for example, under the designation “Schraubentellerfeder” [helical disk springs] by Dr. Werner Röhrs GmbH & Co. KG. They are employed, for example, in hydrogen fuel cells with high energy density, for example of the kind used in satellite space travel or in submarines. Moreover, they can be used in machine tools and tool clamps or in stretch blow-molding machines in PET shaping. The invention is now based on the surprising discovery that the completely different demands of the present use as a joint for an orthopedic device, in comparison to the known uses, are also satisfied by spring elements of this type.

According to the invention, the spring strip has a cross section which has a longer cross-sectional side and, accordingly, a shorter cross-sectional side. The cross section is advantageously rectangular. The four sides form the two longer cross-sectional sides and the two shorter cross-sectional sides, such that the longer cross-sectional side and the shorter cross-sectional side are straight. As an alternative to this, it may also be possible for the cross section to have a curved or arched or irregular configuration, such that the longer cross-sectional sides and/or the shorter cross-sectional sides are themselves curved.

The spring axis runs in the longitudinal direction of the at least two helical springs. According to the invention, the helical springs are intended to be wound with the longer cross-sectional side edgeways to the spring axis. This means in particular that an angle that the longer cross-sectional side encloses with a direction which is perpendicular to the spring axis is preferably between 45° and −45°, preferably between 30° and −30°, particularly preferably between 20° and −20°. In the event that the longer cross-sectional side is not straight, and instead is curved or arched for example, this angle applies in particular at the radially inner end of the longer cross-sectional side relative to the spring axis.

The fact that the helical spring is wound from a spring strip does not mean that it is also produced in this way. This is intended merely to describe the shape of the helical spring. Although helical springs for spring elements for joints according to the present invention are advantageously also produced in this way, it is nonetheless also possible that corresponding helical springs having almost the same properties are produced generically, for example by laser sintering from titanium. In this way, it is also possible to produce contours which cannot be produced by the actual winding of a spring strip.

According to the invention, the at least two helical springs are wound with their longitudinal side edgeways, in such a way that the longer cross-sectional sides at least of one of the helical springs, preferably of both helical springs, have an angle to the spring axis deviating from 90°. The longer cross-sectional sides of the at least two helical springs deviate from the right angle to the spring axis in different directions. This ensures that the at least two helical springs do not bear on each other across the full surface area, as a result of which the spring effect would be greatly impaired or entirely annulled. Particularly advantageously, the two helical springs bear on each other only along one contact line. When the spring is loaded, for example by being pressed together, i.e. compressed, the at least two helical springs are likewise compressed, and the angle of the longer cross-sectional side of the cross section of the respective spring strip of the two helical springs relative to the spring axis changes. In this case, the deviation from the right angle to the spring axis advantageously decreases as the load on the spring elements used here increases.

In principle, it is sufficient if the longer cross-sectional side of only one of the used helical springs has an angle to the spring axis that deviates from 90°. The respective second helical spring used can be designed such that the longer cross-sectional side is arranged exactly at a right angle to the spring axis. Advantageously, however, all of the helical springs used are designed such that the longer cross-sectional sides have an angle to the spring axis deviating from 90°. In this case it is advantageous if the angles of the longer cross-sectional sides for different helical springs deviate in different directions from the right angle to the spring axis and/or deviate to different extents from this right angle. The angles that are actually chosen depend on the required spring force, the spring characteristic and other demands. It is also possible to vary the angle of the longer cross-sectional side to the spring axis along the length of the respective spring element and thereby obtain spring constants of different magnitude in different regions of the respective spring element.

The special nature of the spring element, with two helical springs which are screwed into each other and are advantageously of identical configuration, on the one hand reduces the susceptibility of the spring element to breaking, for example due to high mechanical loads. Since the two helical springs screwed into each other are each formed in one piece and are held in their position by the respective other helical spring, breaking of one of the helical springs does not cause any change of the applied spring force or the release of individual damaged parts. This also avoids the danger of further fractures of other elements or further fractures of both helical springs at other locations. On the other hand, the number of components required is greatly reduced by comparison with a spring arrangement known from the prior art, in particular for large spring excursions, since it is no longer necessary to use a large number of separate disk springs that are to be produced individually and assembled. Regardless of the required length of the respective spring element, all that has to be done is for two helical springs to be screwed into each other, as a result of which the production method is expedited and at the same time the production costs are lowered.

In a preferred embodiment of the joint, the joint has at least two spring elements, such that the second element is pivotable in two opposite directions counter to a force applied by at least one of the at least two spring elements. In this way, for example in the case of an ankle joint that is to be formed, aided or simulated by the joint according to this illustrative embodiment of the present invention, both the plantar flexion and the dorsal flexion can be spring-loaded. Preferably, the at least two spring elements each have at least two helical springs which are each wound from a spring strip having a longer cross-sectional side edgeways with respect to the spring axis and which are screwed into each other in such a way that the longer cross-sectional sides have an angle deviating from 90° relative to the spring axis in different directions, and such that the spring strips bear on each other. In this way, the advantages achieved by this type of spring element can be used twice over. Of course, it is also possible to use more than two helical springs that are screwed into one another. In this way, the spring hardness is further increased while the installation space remains almost the same.

It has proven advantageous if the spring strips are made at least partially from a flat wire or a steel strip. The spring element then has at least in part, but preferably along its entire length, two identical helical compression springs which are screwed into each other, and each of which is wound from a steel strip or from a flat wire with a disk-spring-like cross section edgeways and obliquely with respect to the center axis of the spring element. In the used helical spring, the cross section of the used steel strip or of the used flat wire is then tilted in relation to the longitudinal axis of the helical spring. Advantageously, the helical springs screwed into each other are used such that this tilt in relation to the longitudinal axis of the respective helical spring is present in different directions. In this way, disk-spring-like contact faces of the individual helical springs bearing on each other are obtained. The helical springs can also be produced from titanium or other metals or alloys, in particular with or without iron, carbon or plastic, and can be made of different or identical materials.

In a preferred embodiment, a buffer element, in particular made of an elastomer, particularly preferably made of a polyurethane elastomer such as the one commercially available under the designation “Eladur”, is located in at least one of the spring elements. In the preferred embodiment, the buffer element, which advantageously has a cylindrical shape, is inserted along the longitudinal axis of the spring element into the two helical springs that are screwed into each other. It thus serves as a further damping element and spring element, as a limit stop and as a guide mandrel. Alternatively or in addition to this, a buffer element of this kind can also be arranged in the form of a hollow cylinder, and in another geometric configuration, around the respective spring element. For example, it is conceivable for two, three, four or more cylindrical buffer elements to be arranged on the outer face of the spring element, distributed about the circumference thereof. However, the positioning of the buffer element in the interior of the spring element has the advantage that no additional installation space is needed for this. It is also possible to cast the spring element into the material of the buffer element.

The buffering effect and, if appropriate, the hardness of an impact can be adjusted via the elasticity or hardness of the buffer element which, depending on the material used, can be selected almost freely across a defined range.

It has proven advantageous if the buffer element is shorter than the spring element in or on which it is arranged. In this way, in a particularly preferred embodiment, it can serve as an additional buffer for an impact, in order to additionally prevent a hard impact here. The impact can be damped in this way. Such embodiments moreover have the advantage that the actual and exact position of the impact, and thus the maximum possible pivoting angle of the second element, which can be a foot part for example, relative to the first element, which can be a below-knee part for example, can also be adapted individually to the respective patient in a particularly simple way by an orthopedic technician. The latter simply has to shorten the buffer element accordingly in order to be able to individually adapt the impact and, consequently, the possible maximum pivoting angle in one or both directions. It is thereby also possible, for example in the course of therapy, to modify this maximum possible pivoting angle and therefore the position of the impact, for example by simply using new buffer elements.

Of course, the buffer element can also be made longer than the spring element and, for example, can protrude from the spring element at one end of the spring element. At this location, a recess is preferably provided in the joint, into which recess the protruding part of the buffer element is inserted. The impact effect, the modification of the spring characteristic and the modification of the stiffness of the spring are determined only by the effective length of the buffer element, i.e. the length of the part of the buffer element located in the region of the spring element. By way of the buffer element, it is possible to support a progressive spring characteristic, since the buffer element, for example upon contact with a stop, enters the spaces between the respective spring elements and thus modifies the spring characteristic.

If a buffer element is located in the interior of the spring element, it is advantageous if the external diameter of the buffer element is exactly as large or almost exactly as large as the internal diameter of the spring element. Under strong loads, by which it is shortened, the buffer element is thus pressed into the spaces and cavities between the spring strips of the two helical springs screwed into each other. On the one hand, the impact effect is thereby improved and the stiffness of the spring element increased, and, on the other hand, the durability of the spring element and its useful life are increased.

The joint preferably has at least one tensioning device, with which at least one of the spring elements can be pretensioned. The joint preferably has a tensioning device for each of the spring elements used.

It has proven particularly advantageous if the degree of the pretensioning or the pretensioning force is designed to be adjustable. This can be achieved, for example, by using tensioning elements which already pretension the used spring element with a certain force in a zero position of the respective joint. If the joint has at least two such spring elements whose pretensioning is adjustable, it is thereby also possible to adjust the respective zero position of the joint, i.e. the position that the second element adopts relative to the first element when no additional external forces act on the joint. This is also quite advantageous for therapeutic and/or rehabilitation purposes.

In a preferred embodiment of the joint, the at least two spring elements are configured differently. For example, different helical springs can be used for the respective spring elements, although it has proven advantageous if identical helical springs are screwed into each other within one spring element. By using different spring elements, it is possible, for example, for the pivoting of the second element relative to the first element to be made easier in a first direction than in a second direction counter to the first direction. It is also possible to use spring elements of different length, as a result of which it is possible, for example, to adjust and determine the spring excursion by which the respective spring element can be compressed, for example. In this way, it is likewise possible to adjust the maximum possible pivoting angle in this direction.

As has already been mentioned, the joint is advantageously an ankle joint for a leg orthosis or an ankle orthosis.

In a preferred embodiment of the joint, the at least one spring element is surrounded by a damping material, in particular an elastomer. It is advantageously encapsulated by the latter. This prevents a situation where the helical springs of the at least one spring element form a block. This means that the individual windings of the helical springs bear on each other across the full surface area, such that a further compression of the at least one spring element is no longer possible. The useful life of the at least one spring element is greatly reduced by this and the probability of mechanical failure is increased. The damping material, which is consequently also located between the individual windings of the helical springs within the at least one spring element, advantageously has rubber-elastic properties and is, for example, an elastomer. A damped impact is thus obtained within the at least one spring element, which impact prevents the complete compression of the at least one spring element and at the same time prevents the disadvantages of a fixed stop of the kind used in many orthopedic joints. Fixed stops result in poor wearing and walking comfort, which is prevented by the damped stop provided by the damping material. The at least one spring element can advantageously be completely encapsulated by a plastic or a polymer, for example an elastomer. The Shore hardness can be constant or variable within the damping material.

However, at least one channel, which is not filled with the damping material, is preferably located in the at least one spring element. The already described buffer element is preferably located in this channel. By means of this simple embodiment, the damped stop can be individually adjusted since, on the one hand, the plastic, for example the elastomer, which forms the damping material, and, on the other hand, a plastic, in particular an elastomer, which forms the at least one buffer element, can be selected freely and individually. It is possible to select different Shore hardnesses or other properties, for example elasticities. Of course, it is also possible to provide the channel in the interior of the spring element in which no damping material is located, without a buffer element being located in this channel.

Advantageously, the damping material and the material of the buffer element have different Shore hardnesses.

An illustrative embodiment of the present invention is explained in more detail below with the aid of the accompanying figures, in which:

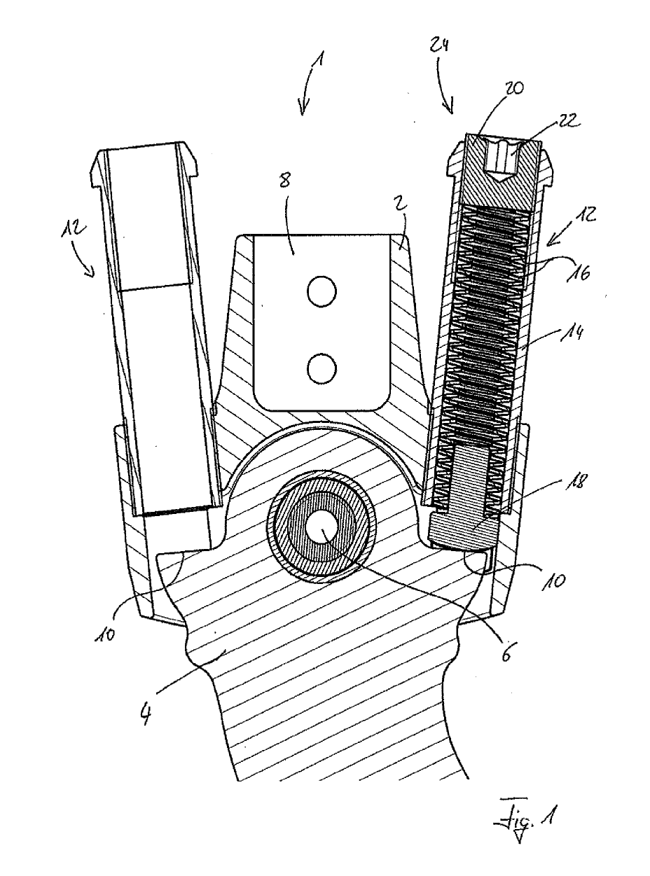

FIG. 1 shows a joint according to a first illustrative embodiment of the present invention,

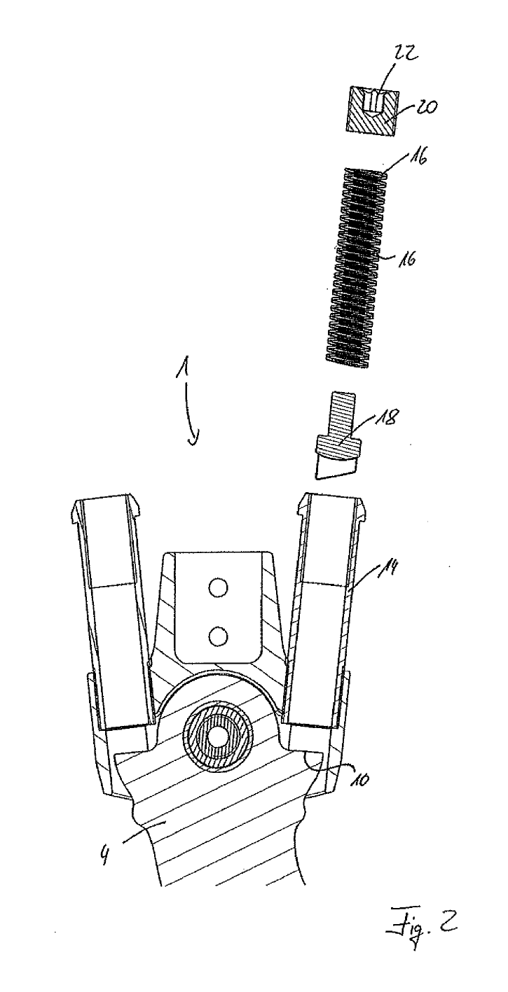

FIG. 2 shows the joint from FIG. 1 in an exploded view,

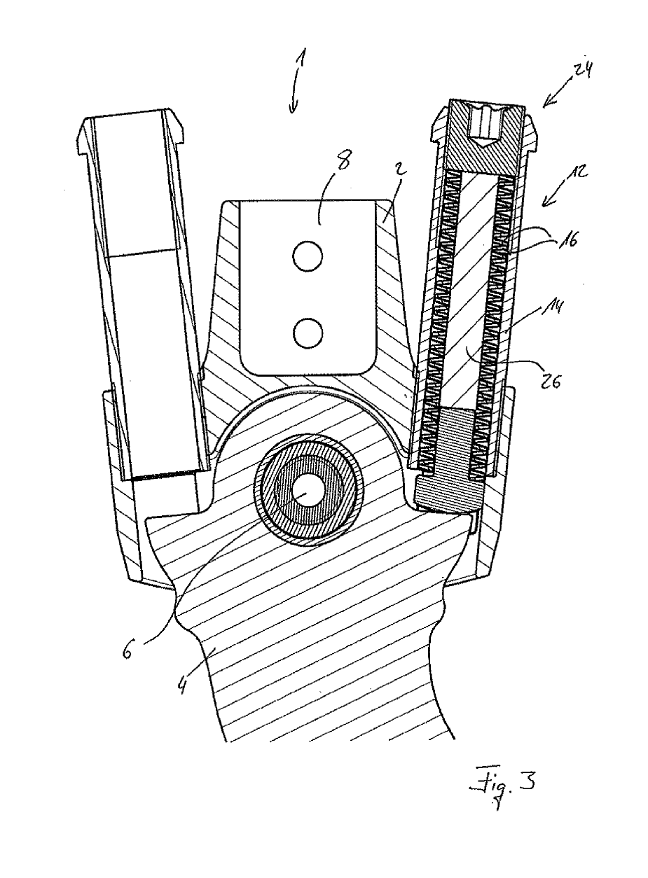

FIG. 3 shows a joint according to a second illustrative embodiment of the present invention,

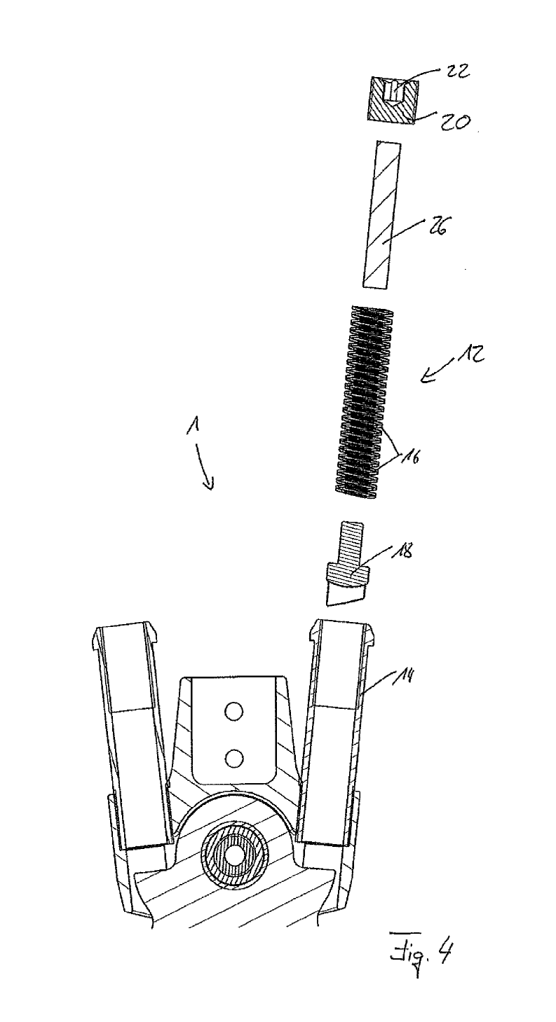

FIG. 4 shows a joint according to the illustrative embodiment from FIG. 3 in an exploded view,

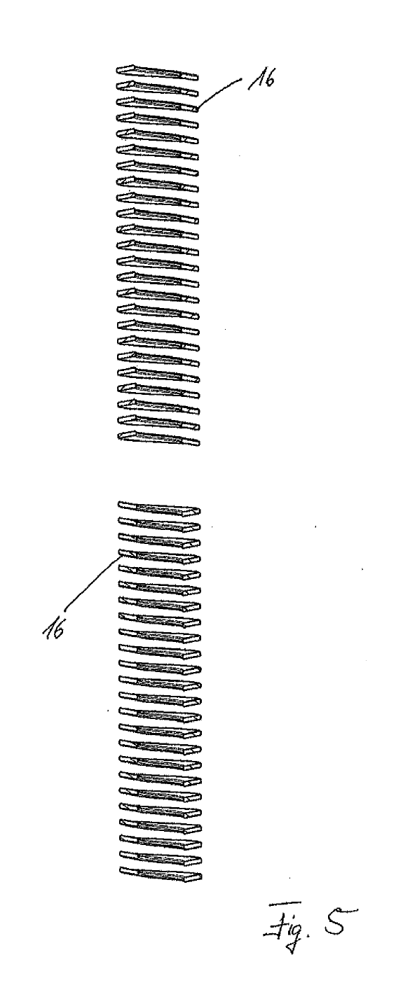

FIGS. 5-7 show cross-sectional views of two helical springs in different stages during the production of a spring element,

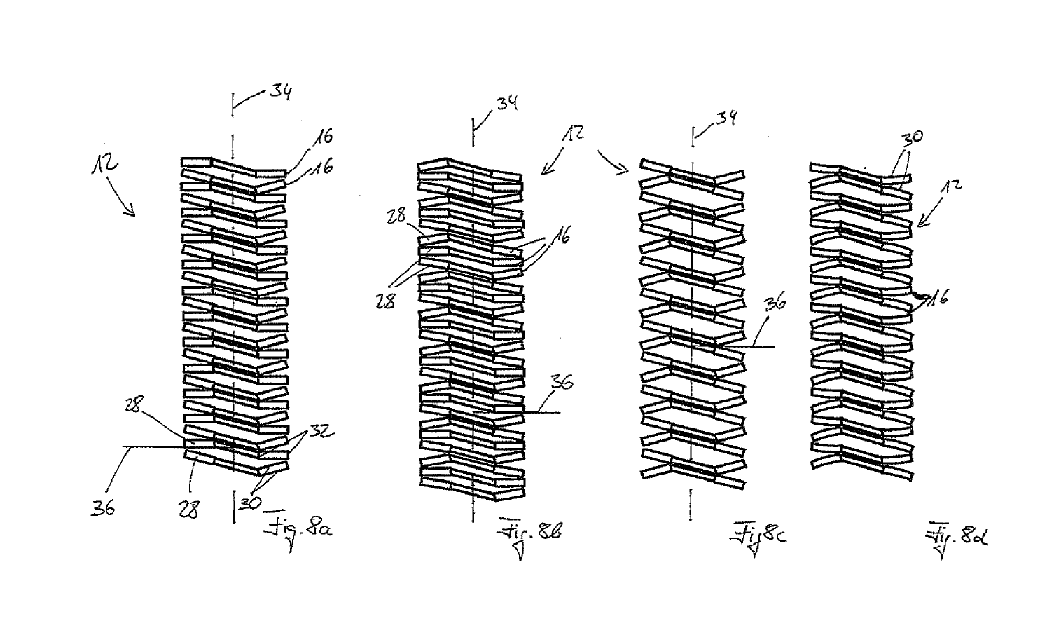

FIGS. 8a-8d show cross sections through different spring elements,

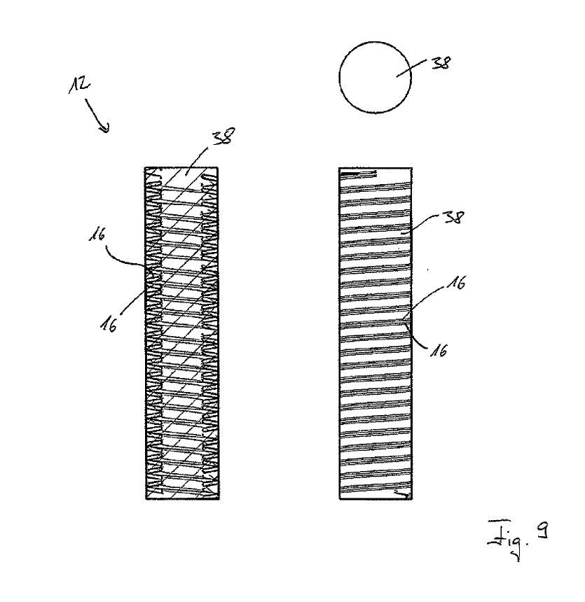

FIGS. 9-12 show differently configured spring elements, in each case in a cross-sectional view (left), a view perpendicular to the spring axis (bottom right), and a view along the spring axis (top right).

FIG. 1 shows a joint 1 according to a first illustrative embodiment of the present invention. It has a first element 2 and a second element 4. The second element 4 is mounted pivotably on the first element 2 about a pivot axis 6. The joint 1 can be an ankle joint, for example. In this case, the second element 4 forms a foot part, while the first element 2 forms a below-knee part. On the first element 2, a receptacle 8 can be seen on which, for example, a rail element of an orthosis can be secured.

The second element 4 has two stop elements 10 which, in the illustrative embodiment shown, are designed as shoulders of the second element 4. The joint 1 has two spring elements 12, of which only the right-hand spring element 12 is shown. It is located in a sleeve 14, by which it is protected from dirt and is at the same time guided. The spring element 12 comprises two helical springs 16 which are screwed into each other. By virtue of the positioning in the sleeve 14 and on account of the inherent stability of the helical springs 16, a further guide, for example through an inner mandrel, is not necessary, although it may be advantageous in some designs.

A counter-stop element 18 is located at what is the lower end of the spring element 12 in FIG. 1. This counter-stop element 18 bears on the stop element 10 of the second element 4. In the upper region of the spring element, a screw element 20 is present which is screwed into an inner thread of the sleeve 14. By way of a depression 22 which is present in the screw element 20 and into which a form-fit element can be introduced, the screw element 20 can be rotated relative to the sleeve 14 and thus screwed farther into or out of the sleeve. In this way, the screw element 20 together with the inner thread of the sleeve 14 becomes a tensioning device 24. If the screw element 20 is screwed farther into the sleeve, the two helical springs 16 and thus the spring element 12 are pressed together. The pretensioning of the helical springs 16 and of the spring element 12 is thereby increased. A pivoting of the second element 4 counterclockwise about the pivot axis 6 is made difficult in this way.

The spring element 12 shown on the left in FIG. 1 is advantageously of identical configuration, although it may be quite advantageous to use different helical springs 16 than in the case of the spring element 12 shown on the right in FIG. 1. These can differ in terms of material, material strength, number of windings and/or length.

FIG. 2 shows the joint 1 in an exploded view. The latter depicts the two helical springs 16, the screw element 20 with the depression 22, and the sleeve 14 into which these components are inserted. Below the helical springs 16, the counter-stop element 18 is shown which serves as contact to the stop element 10 on the second element 4.

FIG. 3 shows a joint 1 according to a second illustrative embodiment of the present invention in a view according to FIG. 1. Here too, the first element 2 with the receptacle 8 is arranged on the second element 4 so as to be pivotable about the pivot axis 6. The spring element 12 with the two helical springs 16 is arranged in the interior of the sleeve 14 and can be pretensioned by the tensioning device 24. In contrast to the illustrative embodiment shown in FIG. 1, a buffer element 26 is now located in the interior of the spring element, which buffer element 26 acts as a guide, additional stop and additional damping element. The length of the buffer element 26 determines when a further compression of the spring element 12 is no longer possible and, consequently, a further pivoting of the second element 4 about the pivot axis 6 relative to the first element 2 is excluded. Here too, the second spring element 12 (not shown) in the left-hand part of FIG. 3 can be designed identically to or differently from the spring element 12. In particular, it is possible to provide such a buffer element 26 only in one of the two spring elements 12.

FIG. 4 shows the joint 1 from FIG. 3 in an exploded view.

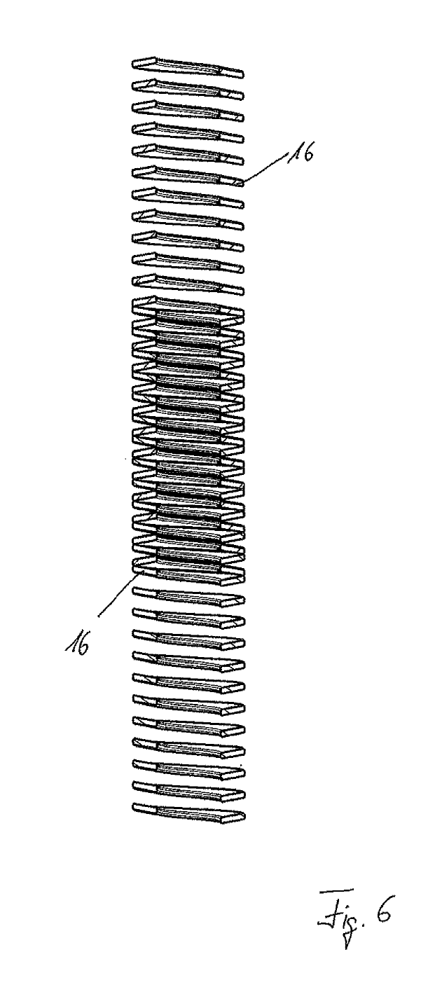

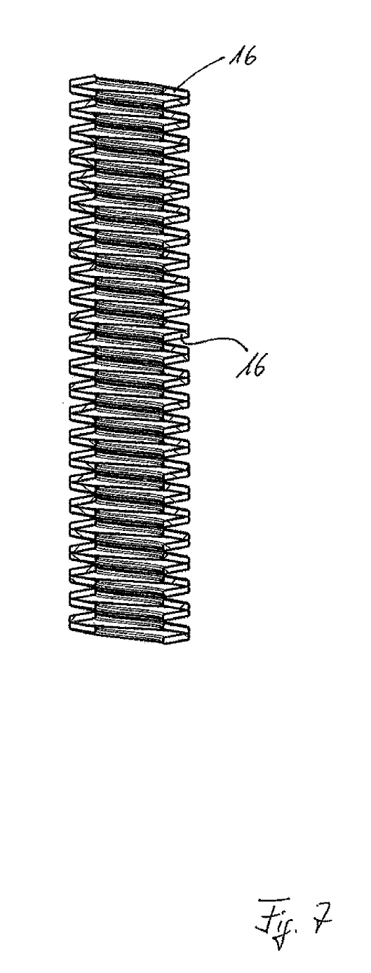

FIGS. 5 to 7 each show a cross-sectional view of two helical springs 16. These are screwed into each other in order to produce a spring element 12. In FIG. 5, the two helical springs 16 are shown spaced apart from each other. It will be seen that both have an almost rectangular cross section and are wound obliquely with respect to a center axis which runs from the bottom upward in FIGS. 5 to 7. The long sides of the almost rectangular cross section thus form an angle different than 90°, wherein this angle for the two helical springs 16 deviates in different directions from the 90° angle with respect to the center axis. This results in a positioning of the two helical springs 16 relative to each other which is similar to the arrangement of separate disk springs. This is shown in the central area of FIG. 6 and in FIG. 7, where the two helical springs 16 are already screwed into each other.

FIGS. 8a to 8d show cross sections through different spring elements.

FIG. 8a shows a spring element 12 which is composed of two helical springs 16 screwed into each other. Each of these helical springs has a cross section 28 which is of rectangular configuration and which thus has two longer cross-sectional sides 30 and two shorter cross-sectional sides 32. FIG. 8a also shows a spring axis 34. It also shows a line 36 running at a right angle to the spring axis 34.

It will be seen that one of the two helical springs 16 has a cross section 28 whose longer cross-sectional sides 30 run exactly parallel to the line 36 and thus enclose an angle of 90° to the spring axis 34. By contrast, the longer cross-sectional sides 30 of the second helical spring 16 run at an angle deviating from 90° with respect to the spring axis 34. The cross sections 28 of the two helical springs 16 bear on each other alternately radially inward and radially outward in a linear contour.

FIG. 8b shows a spring element 12 that has been produced from three helical springs 16. Here too, the cross sections 28 are of rectangular configuration. The cross section 28 of the central helical spring 16 has longer cross-sectional sides 30 which run exactly perpendicular to the spring axis 34 and thus parallel to the line 36. The two other helical springs have cross sections 28 whose longer cross-sectional sides 30 enclose an angle deviating from 90° with the spring axis 34. The angles deviate in different directions from 90°. Therefore, three groups of cross sections 28 of the respective helical springs 16 form in the edge region, i.e. radially outward, and bear radially outwardly on each other.

FIG. 8c shows a spring element that has been produced from two helical springs 16, each of them having cross sections 28 which are of rectangular configuration. The longer cross-sectional sides 30 do not run parallel to the line 36 and therefore do not form a right angle to the spring axis 34. However, the deviations from this 90° angle are different for different helical springs 16. The same applies to the arrangement of two helical springs 16 shown in FIG. 8d, with the difference that the longer cross-sectional sides 30 of the two helical springs used are not configured as straight lines and instead have a curved or arched configuration.

FIG. 9 shows a schematic representation of a spring element 12 in different views. The cross section on the left shows the two helical springs 16, which are completely surrounded by a damping material 38. The side view perpendicular to the spring axis also shows the helical springs 16 and the damping material 38 lying between them. At the top right, there is a schematic representation along the spring axis, showing only the damping material 38.

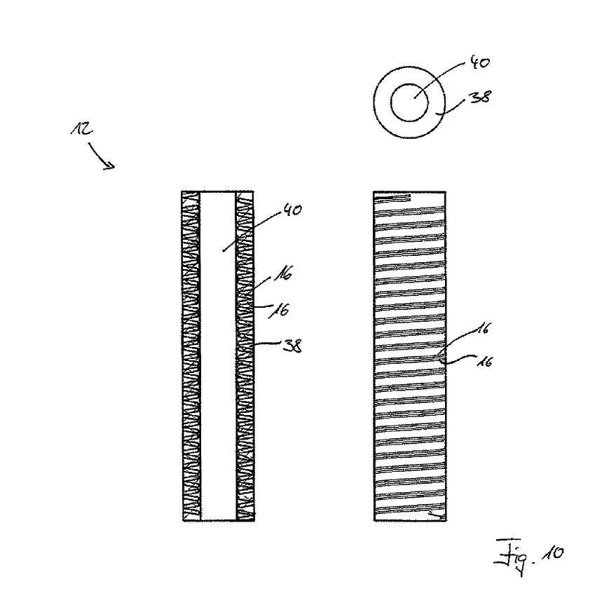

FIG. 10 shows the same views as FIG. 9, but with a modified spring element 12. Here too, the two helical springs 16 are surrounded by the damping material 38. However, a channel 40, which is not filled with damping material 38, is located in the interior of the helical springs 16. The view perpendicular to the spring axis, at the bottom right in FIG. 10, corresponds to the view shown in FIG. 9, since the embodiment of the respective spring element 12 shown in FIG. 9 and in FIG. 10 looks identical in this view. However, at the top right, in the view parallel to the spring axis, the damping material 38 and the channel 40 located centrally therein can be seen. Irrespective of the design, it is not necessary that the channel 40 extends through the entire spring element 12. It is also possible that the channel 40 has an opening only on one side, for example.

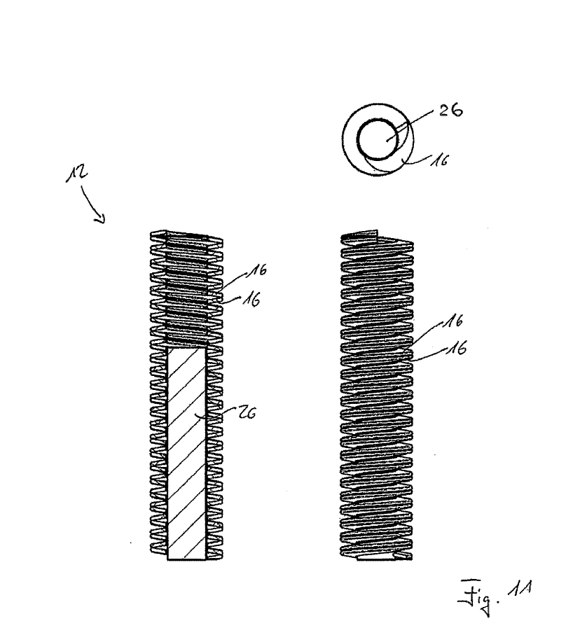

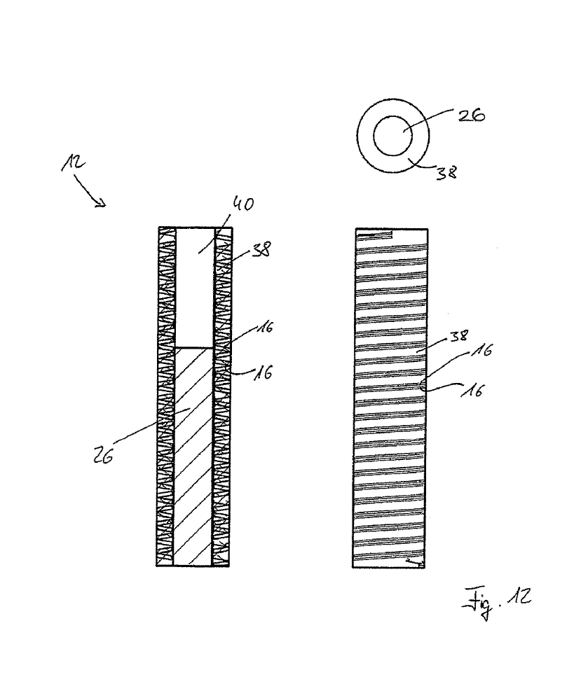

FIG. 11 shows the same views as in FIGS. 9 and 10 with a further spring element 12. The two helical springs 16 can be seen and are not surrounded by a damping material 38. Located in the interior of the helical springs 16 is the buffer element 26, which in particular can also be seen in the view parallel to the spring axis. FIG. 12 shows the view of the spring element 12 shown in FIG. 10, with the buffer element 26 now located in the interior of the channel 40.

### LIST OF REFERENCE SIGNS

1 joint
2 first element
4 second element
6 pivot axis
8 receptacle
10 stop element
12 spring element
14 sleeve
16 helical spring
18 counter-stop element
20 screw element
22 depression
24 tensioning device
26 buffer element
28 cross section
30 longer cross-sectional side
32 shorter cross-sectional side
34 spring axis
36 line
38 damping material
40 channel

1. An orthosis or prosthesis joint for an orthopedic device, comprising:
a first element;
at least one spring element;
a second element which is mounted pivotably on the first element counter to a force applied by the at least one spring element in at least one direction;
wherein the at least one spring element has at least two helical springs, the at least two helical springs:
are each wound from a spring strip having a longer cross-sectional side edgeways with respect to the spring axis, and
are screwed into each other in such a way that
the longer cross-sectional side of at least one of the helical springs has an angle to the spring axis deviating from 90°, and
the spring strips bear on each other.

2. The joint according to claim 1, wherein the joint has at least two spring elements, and the second element is pivotable in opposite directions counter to a force applied by at least one of the at least two spring elements.

3. The joint according to claim 2, wherein the at least two spring elements each have at least two helical springs which are each wound from a spring strip having a longer cross-sectional side edgeways with respect to the spring axis and which are screwed into each other in such a way that the longer cross-sectional side has an angle deviating from 90° relative to the spring axis in different directions, and such that the spring strips bear on each other.

4. The joint according to claim 1, wherein the spring strips are made at least partially from a flat wire or a steel strip.

5. The joint according to claim 1, wherein a buffer element is located in the at least one spring element, the buffer element comprising a polyurethane elastomer.

6. The joint according to claim 5, wherein the buffer element is shorter than the at least one spring element in which the buffer element is arranged.

7. The joint according to claim 1, further comprising at least one tensioning device, with which the at least one spring element is pretensioned.

8. The joint according to claim 7, wherein the pretensioning is adjustable.

9. The joint according to claim 2, wherein the at least two spring elements are configured differently.

10. The joint according to claim 1, wherein the joint is an ankle joint for a leg orthosis or an ankle orthosis.

11. The joint according to claim 1, wherein the at least one spring element is encapsulated by a damping material, the damping material comprising an elastomer.

12. The joint according to claim 11, further comprising a buffer element and at least one channel, the at least one channel is located in the at least one spring element, and the buffer element is located in the at least one channel.

13. The joint according to claim 12, wherein the damping material and the material of the buffer element have different Shore hardnesses.

14. An orthosis or prosthesis joint, comprising:
a first element;
at least one spring element;
a second element pivotally mounted on the first element counter to a force applied by the at least one spring element;
wherein the at least one spring element comprises at least two helical springs, the at least two helical springs each being formed from a spring strip having a longer cross-sectional side edgeways with respect to a spring axis extending along a longitudinal axis of the at least one spring element, the at least two helical springs being screwed into each other in such a way that the longer cross-sectional side of at least one of the helical springs is arranged at an angle relative to the spring axis which deviates from 90°, and the at least two helical springs bear on each other.

15. The joint according to claim 14, wherein the joint comprises at least two spring elements, and the second element is pivotable in opposite directions counter to a force applied by at least one of the at least two spring elements.

16. The joint according to claim 15, wherein the at least two spring elements each comprise at least two helical springs that are each formed from a spring strip having a longer cross-sectional side edgeways with respect to a spring axis extending along a longitudinal axis of the at least one spring element, the at least two helical springs being screwed into each other in such a way that the longer cross-sectional side of at least one of the helical springs is arranged at an angle relative to the spring axis which deviates from 90°, and the at least two helical springs bear on each other.

17. The joint according to claim 14, wherein the spring strips are made at least partially from a flat wire or a steel strip.

18. The joint according to claim 14, wherein a buffer element is located in the at least one spring element, the buffer element comprising an elastomer.

19. The joint according to claim 18, wherein the buffer element is shorter than the at least one spring element in which the buffer element is arranged.

20. The joint according to claim 14, further comprising at least one tensioning device configured to pretension the at least one spring element.
